(19) United States
(12) Patent Application Publication
(10) Pub. No.: US 2007/0254024 A1
(43) Pub. Date: Nov. 1, 2007

(54) Process for manufacturing films

(75) Inventors: Dominique Cade, Colmar (FR); Xiongwei He, Andolsheim (FR)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/788,159

(22) Filed: Apr. 19, 2007

(30) Foreign Application Priority Data
Apr. 21, 2006 (EP) 06290669.8
Apr. 12, 2007 (IB) PCT/IB07/00952

(51) Int. Cl. A61K 9/48; B29C 39/10

(52) U.S. Cl. 424/451; 264/4

(57) ABSTRACT

The present invention relates to a process for manufacturing hard capsules, comprising the steps of dispersing a water-soluble cellulose ether derivative and complete amount of a setting system comprising at least one hydrocolloid or a mixture of hydrocolloids in water having a temperature of 60° C. or above and, if required, adding one or more gelling aids, then subsequently mixing and cooling the dispersion to a temperature of less than 48° C. so as to obtain a film forming mixture, dipping a capsule mould pin in the film forming mixture, inducing gelation of the film forming mixture adhering to the surface of the capsule pin mould, and drying the film obtained. The invention also relates to capsules obtained with such a process.

### FIELD OF THE INVENTION

The present invention relates to a process for preparing a film forming composition, manufacturing hard capsules therefrom, based on water-soluble cellulose ether derivatives and to hard capsules prepared by said process.

### BACKGROUND OF THE INVENTION

For the industrial manufacture of pharmaceutical capsules gelatine is most preferred for its gelling, film forming and surface active properties. The manufacture of hard gelatine capsules by dip moulding process exploits fully its gelling and film forming abilities. Such capsules are manufactured by dipping mould pins into a hot solution of gelatine, removing the pins from the gelatine solution, allowing the gelatine solution attached on pins to set by cooling, drying and stripping the so-formed shells from the pins. The setting of the solution on the mould pins after dipping is the critical step to obtain a uniform thickness of the capsule shell.

Capsules are widely used in the pharmaceutical industry as well as in the health food supplement market. The main usage thereof is as dosage form for solid, semi-solid, liquid, pellet or herbal preparations. A primary objective of these dosage forms is to have a good disintegration after being administrated in order to enable an effective dissolution of the active substances in the appropriate digestive organ. Consequently, this disintegration characteristic has to remain stable over time when finished products are stored prior to use.

The traditional material for forming the capsule shell is gelatine, because it has the correct and quite ideal properties. Nevertheless gelatin has some disadvantages which make it necessary to have other capsule shell materials available.

Cellulose ether derivatives are known as ideal replacement materials for gelatin. It has been tried to use them e.g. on conventional capsule-making machines, but if the cellulose ether derivative is used alone the process needs to be modified because of their temperature related properties. The viscosity of their solutions, when heated from room temperature, first decreases to a minimum value then it increases rapidly to a gel point, which is only a few degrees above the point of minimum viscosity. Various celluloses and manufacturing methods have been used in the past including solvent evaporation to form a film, using thermal gelling process temperatures at temperatures at or above 100° C. and then using moulds heated to 200° C. using PTFE granules.

The first hydroxyalkyl alkyl cellulose capsules, in particular hydroxypropyl methyl cellulose (HPMC) capsules were made by a thermal gelation process. This process is based on the fact that an aqueous solution of HPMC will gel when heated to a certain temperature. The setting is an indispensable requirement in order to achieve a uniform film thickness distribution. Without the setting the solution will flow down from the pins, no capsule can be produced.

For example, EP 0781542 (A2) relates to a method and apparatus for manufacturing pharmaceutical capsules use an aqueous solution of a thermogelling cellulose ether derivative composition and use capsule body pins and capsule cap pins as moulds. The method involves heating the pins, dipping the pins into the solution to cause the solution to gelatinise on the surface of the pins, removing the pins and drying the gelatinised solution on the surface of the pins to form capsule bodies and capsule caps. Pins are heated pre-dip and post-dip to facilitate gelation. Counterflow air is applied to provide drying from the inside and radiant heat is applied from a dome-shelter radiant heater mounted facing the domes of a planar array of pins to ensure the solution is fully gelatinized before drying. It is a disadvantage of this process that the coating on the pins is not gelled or solidified and can fall down from the pins if heating is insufficient or the coating can be wrinkled during gelation if the heating temperature is too high. Furtheron this method requires a special apparatus or operation for heating the moulding pins.

It is evident that with such high pin and drying temperature, the process is difficult to keep stable or consistent. For example, any machine stop will affect seriously the capsule quality.

To overcome the problems and disadvantages associated with thermal gelation methods it has been tried to modify the cellulose ether dipping mixture by using an additional gelling system.

U.S. Pat. No. 5,756,123 relates to a process for manufacturing HPMC capsules wherein the capsule shell being prepared by drying an aqueous solution comprising 18 to 28% by weight of hydroxypropyl-methyl cellulose having a viscosity of 2.4 to 5.4 centistokes as measured in a 2% aqueous solution at 20° C. as a base, 0.01 to 0.09% by weight of carrageenan as a gelling agent, and 0.05 to 0.6% by weight of at least one ion selected from the group consisting of potassium and calcium ions as a co-gelling agent. The process makes use of conventional dipping technique wherein the immersion solution in which shaping pins are immersed is preferably set at a temperature of 48° C. to 55° C., especially 51° C. to 53° C. Outside this temperature range, the immersion solution would have a finely varying jelly viscosity and thickly or thinly adhere to the pins, failing to form shells of uniform gage. Thereafter the immersion solution adhering to the pins is preferably dried at a temperature of 25° C. to 35° C. for 40 to 60 minutes. Through the drying step, the immersion solution adhering to the pins is concentrated to form hard shells around the pins.

U.S. Pat. No. 5,431,917 relates to a method for producing a hard capsule for pharmaceutical drugs, said method comprising the steps of preparing an aqueous solution of a capsule base containing a water-soluble cellulose derivative in the form of a cellulose ether derivative in which some or all of the hydroxyl groups thereof are substituted with an alkyl group and/or a hydroxyalkyl group, a gelatinizing agent and an auxiliary for gelation, immersing a capsule moulding pin in the aqueous solution of the capsule base, subsequently drawing out the moulding pin in the aqueous solution of the capsule base, subjecting the aqueous solution of the capsule base attached to the outer surface of the moulding pin to gelation at a temperature of 22.5° C. to 25.5° C., and forming a capsule film on the outer surface of the moulding pin, wherein the solution temperature of the aqueous solution of the capsule base is 48° C. to 55° C.

At high temperature, the HPMC dipping mixture (dispersion) has very low viscosity as the HPMC substantially does not dissolve. With the cooling, the viscosity increases progressively. From about 55° C., further cooling induces HPMC particles solubilisation, leading a more pronounced increase in the viscosity. At 50° C. the increase in the viscosity becomes so sharp that the mixture turns to a jelly-like solution with an extremely high viscosity. This phenomenon could be of the same mechanism to the thermal gelation, the intermolecular hydrophobic interaction between the high substituent portions where 3 hydroxyl groups are all substituted with the methoxyl groups. The viscosity of jelly-like solution is much higher than the workable range of dipping process and prevents the good adhesion of the solution to the capsule-forming pins during dipping and in particular prevents the manufacturing of capsules on the conventional dip moulding process equipment.

In practice, the dipping temperature is kept higher than 50° C. in order to avoid the above issues. However, experience revealed that this process is not without weakness and difficulties. When the dipping temperature is close to 50° C., such as <55° C., the viscosity of the dipping mixture is in sharp increase with the temperature cooling. Any change in the dipping mixture temperature will lead to important change in viscosity, and consequently to important change in the capsule dimension, which makes problematic the filling operation on the high speed equipment. On the other side, if the dipping temperature is significantly higher than 50° C., the dipping mixture is a dispersion and most HPMC particles are not dissolved in it. These particles swell or partially dissolve only on the dipping pin after the dipping which reduces the temperature of the HPMC dispersion attached on the pin surface, followed immediately by the formation of gelling system network and capsule drying. All these factors make the solubilisation of HPMC particles very limited; the resulted capsules shell is heterogeneous, with low transparency and poor mechanical performance.

EP1044682 relates to method of manufacturing hard capsules which characterized by comprising the steps of dispersing a water-soluble cellulose derivative in hot water and cooling the dispersion to effect dissolution of the water-soluble cellulose derivative in the water, adding and dissolving a gelling agent in the water-soluble cellulose derivative solution to give a capsule-preparing solution, dipping a capsule-forming pin into the capsule-preparing solution at a predetermined temperature, then drawing out the pin and inducing gelation of the capsule-preparing solution adhering to the pin.

However, it is evident that the above process with such cooling and heating back steps is time and energy consuming. Furtheron the long time cooling and heating around 30-40° C. is a very favourable condition for bacteria growth.

On the other hand, there is another issue in the recycling of cellulose ether derivative material which has e.g. already been formed into films or capsules or resulting from the capsule cutting for the target length (trims). In fact, the trims or other recycling materials from defective capsules contain already the gelling system. It will gel when the solution is cooled down to room temperature. And the temperature at which the solution is heated up back (35-50° C.) is not high enough to dissolve the gel formed at room temperature. Consequently is impossible to recover the gelling ability of the gelling agent contained in the trim.

Since the first patents issued many attempts have been made to manufacture cellulose ether capsules in quantity, using different compositions, with sufficient uniformity to be suitable for filling in modern high-speed filling machines. It has also been tried to manufacture films and other containers of cellulose ether derivatives on conventional machines and/or in industrial scale.

In the following this is explained in more detail on basis of capsule manufacturing as an example. Until today, capsules manufactured in quantity suffer imperfections such as haze, wrinkles, starred ends and corrugations, inhomogeneous form, opaqueness. These imperfections result in films or capsules either breaking, failing to separate, providing bad solubilisation or disintegration or jamming in the high-speed filling machine.

Such methods require a strict temperature control of the whole capsule forming process. The setting ability of the film forming mixture, here e.g. the capsule base before immersing the capsule moulding pins, needs to be adjusted so as to obtain uniform capsules. This is disadvantageous both with respect to the equipment and the other resources to be used. To meet these strict temperature control the processes known in the art require a lot of different process steps, e.g. dissolving the cellulose derivative at a certain temperature bringing this mixture to a specific temperature before adding the gelling agent, mixing, addition of further ingredients at specific temperatures and so on. Due to the fact that the entire method is viscosity driven, it is very difficult to find a process window/temperature range where the setting ability of the aqueous capsule base mixture is sufficient, and the ingredients of said mixture are at least partly solubilized. If these conditions are not met, it is impossible to obtain totally homogenous, clear and bubble-free capsule shells. Furthermore, often ageing of the aqueous mixture can become a problem. Aging is a quite general phenomenon which occurs in a wide variety of off-equilibrium materials. Aging affects the properties of materials like mechanical, rheological, magnetic etc. This can for example result in further inhomogeneities.

The present invention has been made under the above-described circumstances, and its object is to provide a process of manufacturing uniform, transparent films useful for hard capsules made of a water-soluble cellulose derivative such as HPMC without requiring a lot of different process steps, easing the requirements of temperature control and reducing length of time. It is an object of the present invention to provide an economical process for manufacturing uniform transparent hard capsules made of water-soluble cellulose ether derivatives wherein the setting ability of the film forming mixture is sufficient; that is, a process which allows the use of conventional film forming or e.g. dip moulding equipment and gives raise to the production of totally homogenous, transparent and bubble-free capsules showing a good dissolution profile, disintegration profile and stability. It is a further object of the invention to provide a more simple process useful for industrial scale manufacturing processes and saving time and energy.

### SUMMARY OF THE INVENTION

A process for manufacturing hard capsules, comprising the steps of
(a) dispersing a water-soluble cellulose ether derivative and complete amount of a setting system comprising at least one hydrocolloid or a mixture of hydrocolloids and if required one or more gelling aid, in water having a temperature of 60° C. or above;
(b) mixing and cooling the dispersion obtained in step (a) to a temperature of less than 48° C. so as to obtain a film forming mixture;
(c) dipping a capsule mould pin in the film forming mixture obtained in step (b);
(d) inducing gelation of the film forming mixture adhering to the surface of the capsule pin mould;
(e) drying the film obtained in step (d).

After drying the capsule it is removed from the surface of the mould pin. The term “capsule” in this process means forming capsule parts (for example, a top portion of a capsule and a bottom portion of a capsule) with the ability to remove the capsule parts from the moulds and then put the parts together to form a whole capsule.

### DESCRIPTION OF THE DRAWINGS

The present invention is illustrated by the Figures in which:

FIG. 1 shows the viscosity evolution with the time of a 17% HPMC dispersion according to Example 1.

FIG. 2 shows the viscosity of the dipping solution as a function of temperature according to Example 2.

FIG. 3 shows the dissolution test results of capsules according to Examples 2 and 4.

FIG. 4 shows the disintegration test results of capsules according to Examples 2 and 4.

FIG. 5 shows the Impact test results of capsules according to Examples 2 and 4.

Further explanation of the Figures is found in the Examples.

### DETAILED DESCRIPTION OF THE INVENTION

In the process of the present invention, first of all a water-soluble cellulose ether derivative and a complete amount of a setting system comprising at least one hydrocolloid and, if required, a gelling aid, are dispersed in hot water. The temperature of the water in step (a) may be selected as appropriate for the type of water-soluble cellulose ether derivative used, although the temperature is preferably in the range of 65 to 85° C., and more preferably in the range of 70 to 85° C. In another preferred embodiment the temperature of the hot water is 70 to 80° C. The water-soluble cellulose ether derivative, the complete amount of a setting system and optionally one or more further ingredients, are added in any given order into hot water preferably of a predetermined temperature within the above mentioned temperature ranges.

With “complete amount of setting system” is meant that all the components of the setting system, including a hydrocolloid, gelling aid and/or sequestering agent, are mixed with the cellulose ether derivative in step (a) of the process. No further addition of setting system takes place after the temperature of the dispersion of step (a) has been brought down to obtain the film forming mixture.

Alternatively, the water-soluble cellulose ether derivative, the complete amount of setting system agent and, optionally, one or more further ingredients, are poured into water having a lower temperature lower than a predetermined temperature, followed by heating to a predetermined temperature within the above mentioned temperature range, i.e. 60° C. or above.

Preferably the dispersion is mixed continuously during cooling. The mixing during cooling has two important roles. The first is to minimize the temperature gradient within the mixture. The second is to avoid the excessively high viscosity observed around temperatures between 40 to 50° C. if the mixture is cooled without mixing or without enough mixing. Preferably the mixing is done in an efficient way in order to achieve the above two effects. However, the mixing should be not too strong in order to avoid the generation of bubbles in the dispersion or film forming mixture.

After the water-soluble cellulose ether derivative and the complete amount of a setting system comprising at least one hydrocolloid and, if required, a gelling aid, have been dispersed in the hot water, the dispersion is cooled to a temperature of less than 48° C. Cooling may be carried out by either natural cooling or forced cooling. Hereby, cooling of the hot dispersion to a temperature of less than 48° C. will result in an optimized aqueous film-forming mixture which is suitable for capsule preparation.

Preferably the mixture is cooled down to less than 48° C. but higher than the setting temperature. The setting temperature is the temperature at which the mixture starts to gel. This can be determined by the sharp increase in the viscosity of the mixture during cooling. A temperature higher than setting temperature avoids the risk of reducing the setting ability by overcooling. This allows in particular the manufacture of hard capsules by conventional dip moulding process. The setting temperature depends on the type of setting system and their relative amounts. Preferably the setting temperature is between 20-45° C.

In this application the terms setting and gelling are used synonymously.

The appropriate cooling time is determined by the effectiveness of cooling without producing a heavy temperature gradient within the mixing vessel and/or causing bubbles in the mixture.

Cooling time preferably is in a range of from 0.5 to 24 hours, more preferably 0.5 to 18 hours, most preferably 1 to 12 hours such as 1 to 8 hours. In general, cooling is carried out to a temperature of less than 48° C., in another embodiment to 47° C. or less and still another embodiment to 45° C. or less.

In an alternative embodiment, cooling of the mixture comprising cellulose ether derivative and setting system is carried out down to a temperature of 35° C. or less, preferably 30° C. or less but above the setting temperature within the time limits given above. Thereafter the aqueous film forming mixture is again heated up to a temperature of below 48° C. so as to obtain the aqueous capsule-preparing film-forming mixture to be used in the subsequent dipping step.

According to the present invention, cooling of the dispersion can be achieved by adding a quenching agent, e.g. water, to the hot dispersion, or providing an external cooling means or any combination of these measures. In a preferred embodiment of the present invention, the external cooling means is a mixer, which can be any mixer well known in the art, e.g. a stirrer.

Thereafter, a capsule mould pin is dipped into the film forming mixture at a predetermined temperature. The contacting temperature, e.g. temperature of the film forming mixture for dipping, may be selected according to the type of water-soluble cellulose ether derivative and the setting system. Preferred is a temperature from higher than 20° C. up to 48° C. In a preferred embodiment of the present invention the temperature is within a range of 40-47.9° C. and, in still another preferred embodiment, the contacting (dipping) temperature is within the range of 43-47.8° C.

In one embodiment of the present invention the capsule mould pin is preconditioned within a specific temperature range. Such a temperature range, for example, may depend on the combination of the film forming mixture as well as on the desired properties of the hard capsule. The temperature of the pin should be less than the setting temperature of the film forming mixture. In a preferred embodiment the temperature of the pin, before contacting it with the film forming mixture, is preferably less than 40° C., more preferably within a range of 15 to 35° C.

In another embodiment of the present invention, a cooled capsule mould pin is used which cooled pin preferably has a temperature in the range of from about 10 to 18° C.

After contacting the capsule mould pin with the film forming mixture, the pin is drawn out of the aqueous film forming mixture, and the film forming mixture adhering to the pin is subjected to gelation and drying, giving a final hard capsule.

The gelation temperature depends mainly on the gelling system and its quantity in the film forming mixture. Gelation of the film forming mixture adhering to the pin can be achieved by cooling on standing. Preferably the drying temperature is in the range of 15-40° C. at the beginning to ensure a quick setting of the mixture attached to the surface of the mould pin and, in a second step, the temperature can be increased to reduce the drying time. However, if the film forming mixture gels under heating, gelation may be effected by heating to a temperature in the range of 50 to 80° C., for example after the pin has been drawn out.

In one embodiment of the present invention, gelation takes place at a temperature between 20 and 47° C. In a more preferred embodiment it takes place between 30-45° C. In yet another embodiment gelation takes place at a temperature in the range of 34-40° C. and, in another particular embodiment, at a temperature between 41-45° C. In another embodiment of the present invention, after drawing out the pin, an initial drying step at a temperature of usually less than 30° C. is carried out and thereafter a second drying step at a temperature usually at a temperature not higher than 40° C. is carried out.

Heating and drying can, for example, be achieved by using air at a corresponding temperature or using IR radiation or the like.

Preferably the final capsule has a water content of 2 to 8% by weight.

For the production of hard capsules by dip moulding process, it is important that the film forming mixture attached to the capsule mould pins after dipping is prohibited from flowing down the pins. Otherwise the obtained film will not have the desired uniform thickness. The setting of the solution on the mould pins after dipping is the critical step to obtain a uniform thickness of the capsule shell. The addition of a setting system to the cellulose ether derivative enables the adaptation of specific and desired gelling properties for a selected process (film forming or dip moulding such as the production of hard HPMC capsules by a conventional dipping process).

The setting system consists of at least one hydrocolloid or mixtures of hydrocolloids and may contain in addition a gelling aid which can be cations and/or sequestering agents.

Non-uniform capsules are until today usually discarded because the current states in the art processes are not suited for recycling these non-uniform capsules. An advantage of the process according to the present invention is that this non-uniform material can be recycled within the process by dispersing it in hot water instead of fresh/new cellulose ether derivative.

In a preferred embodiment of the process according to the present invention, in addition to or instead of fresh cellulose ether derivative as starting material, “recycling” cellulose ether derivative material which has e.g. already been formed into films or capsules (“trims”) can be recycled and re-used in the process claimed according to the present invention.

An advantage of the process according to the present invention is that the trims can be recycled within the process by dispersing it in hot water instead of fresh/new cellulose ether derivative. As the solution is never cooled down to below the gelling temperature, the total gelling ability of the gelling agent already contained in the recycling materials is totally recovered.

In the process claimed according to the present invention a water-soluble cellulose ether derivative, the complete setting system comprising at least one hydrocolloid and, if required, a gelling aid, are dispersed in hot water.

Suitable water-soluble cellulose ether derivatives that can be used in the present invention include alkyl- and/or hydroxyalkyl substituted cellulose ether derivatives, and mixtures thereof wherein the alkyl groups have 1 to 4 carbon atoms in the alkyl chains. Particular examples include methyl cellulose, hydroxyethyl cellulose, hydroxypropyl cellulose, hydroxyethylmethyl cellulose, hydroxyethylethyl cellulose, hydroxypropylmethyl cellulose (HPMC) and mixtures of two or more of the foregoing. Other similar compositions may also be used. Especially preferred is HPMC.

The amount of water-soluble cellulose ether derivative to be used according to the present invention, given as the concentration in the aqueous capsule-preparing mixture, is preferably from 8 to 35% by weight, more preferably 15 to 28% by weight based on the weight of the aqueous capsule-preparing mixture. The amount of the water-soluble cellulose ether derivative or mixture of cellulose ether derivatives in the final capsule is preferably 92 to 98% by weight based on the weight of the capsule. The viscosity of the cellulose ether derivative or blend usually is in the range of 3 to 15 cps in 2% aqueous solution at 20° C., preferably 3 to 10 cps and especially 6 cps.

The setting system to be used according to the present invention may be any substance that induces the gelation of the water-soluble cellulose ether derivative used according to the present invention. Suitable examples include a hydrocolloid or mixtures of hydrocolloids.

Suitable hydrocolloids or mixtures producing synergistic properties may be selected from natural seaweeds, natural seed gums, natural plant exudates, natural fruit extracts, biosynthetic gums, gelatines, biosynthetic processed starch, cellulosic materials and mixtures of two or more of the foregoing. Preferred hydrocolloids to be used according to the present invention are polysaccharides.

The preferred polysaccharides according to the present invention are alginates, agar gum, guar gum, locust bean gum (carob), carrageenan, tara gum, gum arabic, ghatti gum, Khaya grandifolia gum, tragacanth gum, karaya gum, pectin, arabian (araban), xanthan, gellan, starch, Konjac mannan, galactomannan, fucoidan, funoran, and other exocellular polysaccharides. Preferred are exocellular polysaccharides.

The preferred exocellular polysaccharides are xanthan, gellan, carrageenan, furcelleran, succinoglycan, scleroglycan, schizophyllan, tamarind gum, curdlan, pectin, locust bean gum (carob), Konjac mannan, gelatin and dextran. Carrageenan can be k-carrageenan, i-carrageenan or λ-carrageenan or mixtures thereof. Preferred are kappa carrageenan and iota-carrageenan.

In one embodiment of the present invention a more preferred hydrocolloid to be used is carrageenan, pectin, gellan gum and combinations of two or more of the foregoing.

The amount of the hydrocolloid is preferably less than 3%, especially 0.01 to 1% by weight of the aqueous film forming mixture.

Optionally, in addition to water-soluble cellulose ether derivative and hydrocolloid(s), according to the present invention a gelling aid can be added to the mixture to be dispersed in hot water. The gelling aid is selected according to the type of hydrocolloid used. An appropriate combination of hydrocolloid(s) and gelling aid according to the present invention may be called a setting system.

Usually, as a gelling aid there may be used a water-soluble compound that provides cations, preferably mono- or divalent cations such as potassium ions, ammonium ions or calcium ions. The cations are preferably selected from the group consisting of K⁺, Na⁺, Li⁺, NH₄⁺, Ca²⁺ and Mg^{2 +}. Illustrative examples thereof include potassium chloride, potassium acetate, ammonium chloride, ammonium acetate and calcium chloride.

The amount of cations is preferably less than 3%, more preferably less than 1%, and especially less than 0.5% by weight in the aqueous film forming mixture. Suitable ranges are 0.01 to 3%, and especially 0.01 to 1% by weight in the aqueous film forming mixture.

Among the setting systems which preferably used according to the present invention, the systems comprising carrageenan with cation or gellan gum with cation are specifically preferred. And the preferred cations are K⁺, Li⁺, Na⁺ or NH₄⁺. Suitable counterions are acetate, sulphate, carbonate or halogen, e.g. Cl⁻, Br⁻, I⁻.

In a preferred embodiment of the present invention the setting system consists of gellan, more preferably together with a gelling aid. In another preferred embodiment the setting system consists of carrageenan and a cation, preferably potassium. While for carrageenan monovalent cations are preferred, for gellan divalent cations are preferred. Preferred divalent cations to be used as a gelling aid in the presence of gellan are Ca⁺⁺ and/or Mg⁺⁺. In another preferred embodiment a mixture of gellan and carrageenan is used as a setting system, more preferably including a cation, e.g. K⁺, as a gelling aid. In still another preferred embodiment pectin is used as a setting system. When pectin is used as a setting system divalent ions are preferred as gelling aid—preferably Ca⁺⁺.

Optionally, the film forming mixture contains one or more sequestering agents. The preferred sequestering agents to be used according to the present invention are ethylenediaminetetraacetic acid, acetic acid, boric acid, citric acid, edetic acid, gluconic acid, lactic acid, phosphoric acid, tartaric acid or salts thereof, metaphosphates, dihydroxyethylglycine, lecithin or beta cyclodextrin and combinations thereof. Especially preferred is ethylenediaminetetraacetic acid or salts thereof or citric acid or salts thereof. The sequestering agent may also include a mixture of different sequestering agents. The amount of sequestering agent is preferably less than 3%, especially less than 1% by weight based on the weight of the aqueous film forming mixture.

Furthermore, one or more additional ingredients may be added to the dispersion and/or during the cooling step and/or before contacting the film forming mixture with the surface of the shaping device. This addition is not limited to a specific stage of the process as long as it is added before contacting a surface of a shaping device with the film forming mixture. These further ingredient(s) can be added all at the same stage or each one at a different stage as well as parts of the same ingredient can be added at different stages.

In a preferred embodiment of the present invention all ingredients are added at the stage when dispersing the cellulose ether derivative and the setting system.

The optional ingredients may include one or more ingredients selected from the group consisting of colouring agent, surfactant, dissolution enhancer, plasticizer and/or further additives.

In addition, a pharmaceutically acceptable or food grade colouring agent may be present in the mixture to be dispersed in hot water or may be added at a later stage.

The colouring agents may be selected from azo, quinophthalone, triphenylmethane, xanthene—or indigoid dyes; iron oxides or hydroxides; titanium dioxide or natural dyes or mixtures of tow or more of the foregoing. Examples are patent blue V, acid brilliant green BS, red 2G, azorubine, ponceau 4R, amaranth, D+C red 33, D+C red 22, D+C red 26, D+C red 28, D+C yellow 10, yellow 2 G, FD+C yellow 5, FD+C yellow 6, FD+C red 3, FD+C red 40, FD+C blue 1, FD+C blue 2, FD+C green 3, brilliant black BN, carbon black, iron oxide black, iron oxide red, iron oxide yellow, titanium dioxide, riboflavin, carotenes, anthocyanines, turmeric, cochineal extract, chlorophyllin, canthaxanthin, caramel and betanin.

If present, the colouring agent may be contained in an amount in the range of from 0 to 5% by weight of the final capsule.

Furthermore, the final film forming mixture may comprise a surfactant. Suitable surfactants to be used according to the present invention may include silicates, silicones, lecithins and mono- and diglycerides. Substances which function in a similar manner may also be used. The surfactant may be selected from the group consisting of sodium lauryl sulphate (SLS), dioctyl sodium sulfosuccinate (DSS), benzalkonium chloride, benzethonium chloride, cetrimide (trimethyl-tetradecylammonium bromide), fatty acid sugar esters, glyceryl monooleate, polyoxyethylene sorbitan fatty acid esters, polyvinyl alcohol, dimethylpolysiloxan, sorbitan esters, lecithin and mixtures of two or more of the foregoing. Said surfactants preferably are present in the mixture to be dispersed in hot water, in an amount of up to about 0-5%, preferably 0-3% by weight, based on the aqueous film forming mixture. In one embodiment the amount is in the range of 0.1% to 5% by weight, and more preferably between about 0.1% and 3%, by weight, based on the aqueous film forming mixture.

Additional ingredients may include a plasticizer. The plasticizer to be used according to the present invention hereby may include an extremely wide variety of water-soluble or water-miscible plasticizers and mixtures of plasticizers including those selected from the group consisting of glycerol, propylene glycol, monoacetin, diacetin, triacetin, glycol diacetate, hydroxypropylglycerol (and all analogous polyols and the esters or ether derivatives thereof), trimethyl citrate, triethyl citrate, di-n-butyl tartrate and mixtures of two or more of the foregoing. Other carboxylic acid derivatives may also be used.

In order to obtain an even film, the plasticizer is preferably present in a concentration at which the dispersion does not coagulate. The plasticizer should also have a relatively high boiling point so that there is little plasticizer evaporation and the capsules retain their elasticity and softness.

In one preferred embodiment, the plasticizer or mixture of plasticizers used according to the present invention is selected from the group consisting of polyethylene glycol, glycerol, sorbitol, sucrose, corn syrup, fructose, dioctyl-sodium sulfosuccinate, triethyl citrate, tributyl citrate, 1,2-propylenglycol, mono-, di- and triacetates of glycerol, and natural gums. More preferred are glycerol, polyethylene glycol, propylene glycol, citrates and combinations of two or more of the foregoing.

The amount of plasticizer depends on the final application. For hard film formulations, such as for hard capsules, the plasticizer is usually contained in an amount of 0 to 20 wt. % in the final capsule.

Plasticizers to be used according to the present invention may also include one or more members selected from the group consisting of low molecular weight poly(alkylene oxides) (such as, for example, poly(ethylene glycols), poly(propylene glycols), poly(ethylene-propylene glycols)), organic plasticizers of low molecular mass (such as, for example, glycerol, pentaerythritol, glycerol monoacetate, diacetate and triacetate), propylene glycol, sorbitol, sodium diethylsulfosuccinate, triethyl citrate, tributyl citrate, and Na citrate. Also, other substances which function in a similar manner may be used.

In addition, according to the present invention the final film forming mixture may comprise further or other additives such as extenders, fillers, flavouring agents, stabilizers etc.

In a preferred embodiment of the present invention a process for manufacturing capsules is provided wherein a soluble cellulose ether derivative (preferably HPMC), the complete amount of setting system (which is, for example, gellan), and optionally a gelling aid (preferably potassium acetate (KAc)), are dispersed in hot water. In another preferred embodiment the process for manufacturing capsules comprises the dispersion of a soluble cellulose ether derivative (preferably HPMC), the complete amount of setting system (for example, a mixture of gellan and carrageenan), and optionally a gelling aid (preferably KAc), in hot water. In another preferred embodiment the process for manufacturing capsules comprises the dispersion of a soluble cellulose ether derivative (preferably HPMC), the complete amount of setting system (for example, carrageenan) and optionally a gelling aid (preferably KAc), in hot water. Preferably when gellan is used as the setting agent, EDTA is added during the above mentioned processes as a sequestering agent. More preferably, EDTA is added during the preparation of the dispersion in hot water.

While the viscosity of the aqueous film forming mixture is not particularly limited, there are special ranges of interest. For example, one particular embodiment has a range of 100 to 10000 mPa·s, and especially 1000 to 8000 mPa·s, at the capsule dipping temperature. In one preferred embodiment, the viscosity of the mixture mentioned above is in the range of 300-5000 mpa·s. This viscosity is the value obtained using a Brookfield-type rotational viscometer, for example at the capsule dipping temperature.

The process according to the present invention may further comprise a coating step wherein the final capsule is coated. The coating can be e.g. spray coating. The capsules, obtained according to the present invention or the final products thereof may be subsequently coated with a suitable coating agent like cellulose acetate phthalate, polyvinyl acetate phthalate, methacrylic acid polymers, hypromellose phthalate, hydroxypropylmethyl cellulose phthalate, hydroxyalkyl methyl cellulose phthalate or mixtures thereof to provide e.g. enteric properties.

The capsules, obtained according to the present invention may be used for providing unit dosage forms for example for agrochemicals, seeds, herbs, foodstuffs, dyestuffs, pharmaceuticals, flavouring agents and the like.

The film forming mixture manufactured by a process of the present invention can also be used for casting flat films or spray coating or as a sealing liquid for e.g. capsule halves.

The improved properties of the capsules, like good transparency, good dissolution profile, good disintegration profile, good mechanical performance, etc, prepared according to the present invention are demonstrated by the following examples.

### EXAMPLES

The following examples are offered as illustrative of the invention and are not to be construed as limitations thereon. In the Examples and elsewhere in the description of the invention, chemical symbols and terminology have their usual and customary meanings. Temperatures are in degrees C. unless otherwise indicated. Unless otherwise indicated, “water” means purified, distilled and/or de-ionized water. As is true throughout the application, the amounts of the components are in weight percents based on the standard described; if no other standard is described then the total weight of the composition is to be inferred. Capsule sizes specified are those used in the art. HPMC used in the examples has a viscosity of 6 mPas of an 2% aqueous solution at 20° C.

### Example 1

### HPMC Dispersion without Setting System

This example illustrates the role of mixing during cooling of the hot HPMC dispersion in order to avoid forming of a jelly-like solution and the excessive viscosity building.

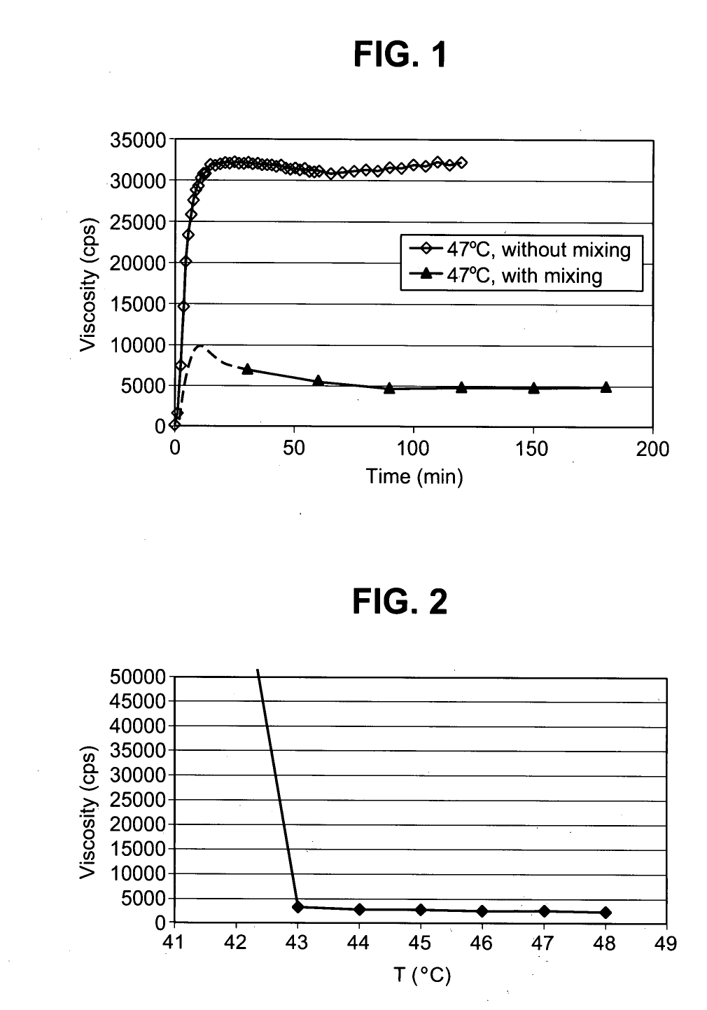

FIG. 1 illustrates the viscosity evolution with the time after a 17% HPMC dispersion is quenched to 47° C. The viscosity is measured by Brookfield type viscometer.

Above results illustrate clearly that without mixing, a jelly-like solution is obtained with an excessively high viscosity which is far beyond the workable viscosity range for dipping process; whereas the viscosity remains at quite acceptable range when with mixing.

### Example 2

### HPMC with Setting System

In a reactor, 4.15 kg of purified water is heated to above 70° C., then 2 grams (0.04% in the final dipping solution) of EDTA disodium, 7 grams (0.14%) of potassium acetate and 3.5 grams (0.07%) of gellan gum are dissolved in the water under mixing, followed by the dispersing of 850 grams (17%) of HPMC powder. After the total dispersing of HPMC powder, reduce the mixing to allow the debubbling of the obtained dispersion. Finally the dispersion is cooled down to 47° C. under constant mixing. A quite strong mixing is preferred during the cooling as far as it does not create bubbles.

FIG. 2 illustrates that the obtained dipping solution of the present example has a setting (gelling) temperature of 43° C., and its viscosity is within the workable viscosity range at temperature between 43-48° C. which is the possible dipping temperature window of the current formula.

The obtained dipping solution is filled into a dipping dish of a hard gelatin capsule manufacturing pilot equipment. A high quality of hard HPMC capsules are produced with similar process conditions to hard gelatin capsule manufactured while keeping the dipping solution in the dipping dish at 47° C.

### Example 3

### HPMC with Setting System with Quenching Step

In a reactor, 3.0 kg of purified water is heated to above 70° C., then 2 grams of EDTA disodium, 7 grams of potassium acetate and 3.5 grams of gellan gum are dissolved in the water under mixing, followed by the dispersing of 850 grams of HPMC powder and the debubbling of the obtained dispersion. The dispersion is then cooled down to 47° C. under constant mixing. At the beginning of the cooling, 1.15 kg of purified water at room temperature is added into the reactor in order to accelerate the cooling.

The dipping solution thus obtained has the same behaviour as the composition of Example 2. Hard HPMC capsules of the same quality as those in Example 2 are produced under the same conditions as in Example 2.

### Example 4

### HPMC with Setting System with Using Recycled Material

In a reactor, 4.15 kg of purified water is heated to above 70° C., then 1 gram of EDTA disodium, 3.5 grams of potassium acetate and 1.75 gram of gellan gum are dissolved in the water under mixing, followed by the dispersing of 425 grams HPMC powder and 430 grams of hard HPMC capsule trims from Example 2. The dispersion obtained is then mixed with a high shearing mixer (Ultra-Turax) during 5 minutes to reduce the trim particle size and to facilitate the dissolving of the gellan gum already present in the recycling trim. After debubbling, the dispersion is cooled down to 47° C. as in Example 2.

The dipping mixture thus obtained has the same composition and behaviour as that in Example 2. Hard HPMC capsules of the same quality as those in Example 2 are produced under the same conditions as in Example 2.

Capsule Performances
Transparency

The following results illustrate the high transparency of the hard HPMC capsules of the current invention.

| Capsule | Transparency |
|---|---|
| Example 2 | 80% |
| Example 4, with 50% trim recycling | 79% |
| Hard gelatin capsule | 81% |

The transparency is measured by a photo spectrometer at 650 nm on the bodies of size 0 capsules.
Dissolution

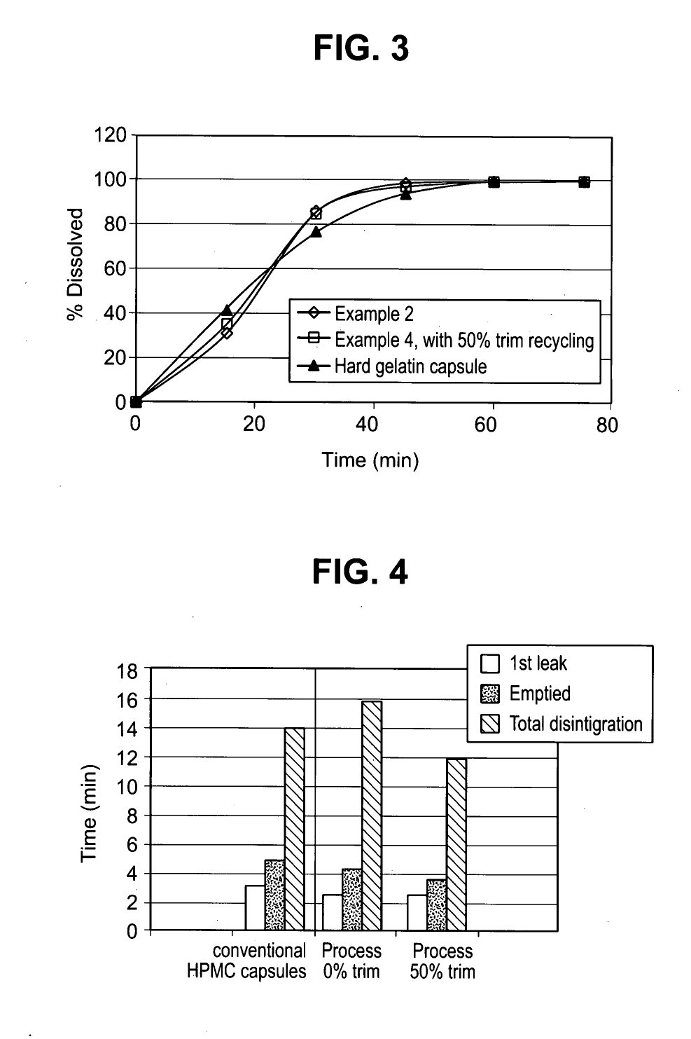

Dissolution test results of capsules filled with acetaminophen in deionised water at 37° C. (USP XXIII dissolution) are represented FIG. 3.
Disintegration

The disintegration behaviour has been compared with conventional HPMC capsules at a pH of 1.2 at 37° C. The results are shown in FIG. 4.
Mechanical performance

Capsule mechanical performance is evaluated by an impact test. This test consists of impacting empty capsules in pre-locked position one by one with a weight of 100 g dropping from a height of 80 mm. 50 capsules were used for each sample, and from the number of broken capsules, the % broken is determined. Before the impact test, the capsules were previously equilibrated at different relative humidity at 22° C.

The results confirmed that the capsules from Examples 2 & 4 are not brittle at all even at extremely low relative humidity conditions.

| | Impact test | | | | | |
|---|---|---|---|---|---|---|
| | | | | | Standard hard | |
| Equilibrium | Example 2 | | Example 4 | | gelatin capsule | |
| relative | | Water | | Water | | Water |
| humidity at | % | content | % | content | % | content |
| 22° C. | Broken | (%) | Broken | (%) | Broken | (%) |
| 2.5% RH | 0 | 1.0 | 0 | 0.8 | 20 | 8.5 |
| 10% RH | 0 | 1.5 | 0 | 1.7 | 10 | 10.2 |
| 23% RH | 0 | 3.0 | 0 | 3.0 | 6 | 11.4 |
| 33% RH | 0 | 4.0 | 0 | 4.0 | 2 | 12.3 |
| 45% RH | 0 | 5.4 | 0 | 5.6 | 2 | 12.9 |

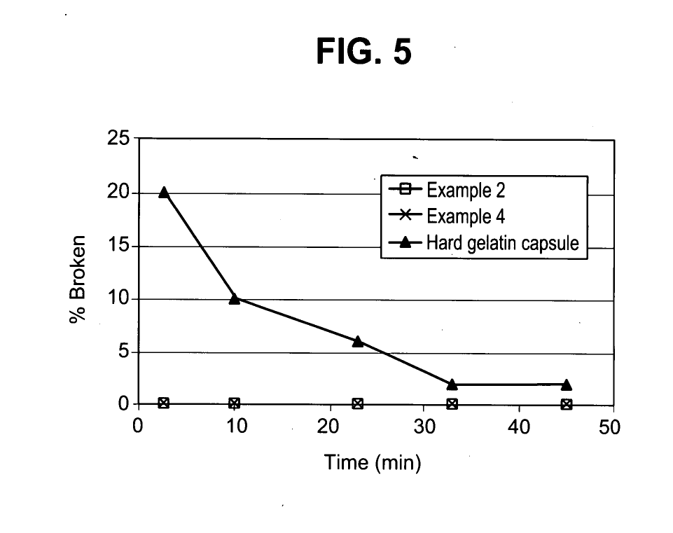

The results are shown in FIG. 5.

1. A process for manufacturing hard capsules, comprising the steps of:
(a) dispersing a water-soluble cellulose ether derivative and a setting system wherein the setting system comprises (i) at least one hydrocolloid or a mixture of hydrocolloids in water having a temperature of 60° C. or above and, optionally (ii) one or more gelling aids;
(b) mixing and cooling the dispersion obtained in step (a) to a temperature of less than 48° C. so as to obtain a film forming mixture;
(c) dipping a capsule mould pin in the film forming mixture obtained in step (b);
(d) inducing gelation of the film forming mixture adhering to the surface of the capsule pin mould; and
(e) drying the film obtained in step (d) to obtain a hard capsule.

2. The process of claim 1, wherein the cellulose ether derivative is selected from the group consisting of alkyl- and hydroxyalkyl substituted cellulose ethers and mixtures thereof wherein the alkyl chains have 1 to 4 carbon atoms.

3. The process of claim 1 wherein the water-soluble cellulose ether derivative is selected from the group consisting of methyl cellulose, hydroxyethyl cellulose, hydroxypropyl cellulose, hydroxyethylmethyl cellulose, hydroxyethylethyl cellulose, hydroxypropylmethyl cellulose (HPMC), and mixtures of two or more of the foregoing.

4. The process of claim 3 wherein the water-soluble cellulose ether derivative is hydroxypropyl methyl cellulose.

5. The process of claim 1, wherein the hydrocolloid is selected from the group consisting of alginates, agar gum, guar gum, locust bean gum (carob), carrageenan, tara gum, gum arabic, ghatti gum, Khaya grandifolia gum, tragacanth gum, karaya gum, pectin, arabian (araban), xanthan, gellan, starch, Konjac mannan, galactomannan, fucoidan, funoran, and other exocellular polysaccharides.

6. The process of claim 1, wherein the hydrocolloid is selected from the group consisting of xanthan, gellan, carrageenan, furcelleran, succinoglycan, scleroglycan, schizophyllan, tamarind gum, curdlan, pectin, locust bean gum (carob), Konjac mannan, gelatin and dextran.

7. The process of claim 6, wherein the hydrocolloid is selected from the group consisting of kappa-carrageen, iota-carrageenan, lambda-carrageenan and mixtures of two or more of the foregoing.

8. The process of claim 1, wherein the gelling aid is a water-soluble compound that provides cations selected from the group consisting of potassium chloride, potassium acetate, ammonium chloride, ammonium acetate and calcium chloride.

9. The process of claim 1, wherein the gelling aid is a cation selected from the group consisting of K⁺, Na⁺, Li⁺, NH₄⁺, Ca⁺⁺ and Mg⁺⁺.

10. The process of claim 1, wherein the setting system further comprises a sequestering agent or mixture of sequestering agents, selected from the group consisting of ethylenediaminetetraacetic acid, acetic acid, boric acid, citric acid, edetic acid, gluconic acid, lactic acid, phosphoric acid, tartaric acid or salts thereof, methaphosphates, dihydroxyethylglycine, lecithin, beta cyclodextrin and mixtures of two or more of the foregoing.

11. The process of claim 1, wherein the water in step (a) has a temperature in the range of 65 to 85° C.

12. The process of claim 1, wherein the dispersion is cooled in step (b) to a temperature below 48° C. but higher than the setting temperature of the setting system.

13. The process of claim 1, wherein the dispersion is cooled to a temperature below 48° C. within 1-8 hours.

14. The process of claim 11, wherein the setting temperature is in the range of 20 to 47° C.

15. The process of claim 12 wherein the setting temperature is in the range of 30 to 45° C.

16. The process of claim 1, wherein the temperature of the film forming mixture in step (c) is in the range of 25-47.9° C.

17. The process of claim 1 comprising the additional step of adding a potion of trim to the dispersion.

18. Hard capsules obtained by a process as claimed in claim 1.
